(19) United States
(12) Patent Application Publication
(10) Pub. No.: US 2022/0289674 A1
(43) Pub. Date: Sep. 15, 2022

(54) A PROCESS FOR PRODUCING 4,4'-DICHLORODIPHENYL SULFONE

(71) Applicant: BASF SE, Ludwigshafen am Rhein (DE)

(72) Inventors: Frauke THRUN, Ludwigshafen am Rhein (DE); Jessica Nadine HAMANN, Ludwigshafen am Rhein (DE); Christian SCHUETZ, Ludwigshafen am Rhein (DE); Oliver BEY, Ludwigshafen am Rhein (DE); Stefan BLEI, Ludwigshafen am Rhein (DE)

(21) Appl. No.: 17/635,033

(22) PCT Filed: Aug. 20, 2020
(86) PCT No.: PCT/EP2020/073356

§ 371 Date: Feb. 14, 2022

(30) Foreign Application Priority Data
Aug. 27, 2019 (EP) 19193690.5

(51) Int. Cl. C07C 315/02; C07C 315/06

(52) U.S. Cl. CPC C07C 315/02; C07C 315/06; C07B 2200/13

(57) ABSTRACT

The invention relates to a process for producing 4,4′-dichlorodiphenyl sulfone, comprising: (a) reacting 4,4′-dichlorodiphenyl sulfoxide and an oxidizing agent in at least one carboxylic acid as solvent to obtain a reaction mixture comprising 4,4′-dichlorodiphenyl sulfone and carboxylic acid; (b) separating the reaction mixture into a first stream comprising 4,4′-dichlorodiphenyl sulfone and a second stream comprising carboxylic acid; (c) purifying the second stream comprising carboxylic acid by distilling a part of the second stream comprising carboxylic acid stripping low boilers from at least a part of the second stream comprising carboxylic acid (d) recycling the purified carboxylic acid into the reaction (a).

The invention relates to a process for producing 4,4′-dichlorodiphenyl sulfone by reacting 4,4′-dichlorodiphenyl sulfoxide with an oxidizing agent in at least one carboxylic acid as solvent and separating, purifying and recycling the at least one carboxylic acid.

4,4′-dichlorodiphenyl sulfone (in the following DCDPS) is used for example as a monomer for preparing polymers like polyether sulfone or polysulfone or as an intermediate of pharmaceuticals, dyes and pesticides.

DCDPS for example is produced by oxidation of 4,4′-dichlorodiphenyl sulfoxide which can be obtained by a Friedel-Crafts reaction of thionyl chloride and monochlorobenzene as starting materials in the presence of a catalyst, for example aluminum chloride.

CN-A 108047101, CN-A 102351758, CN-B 104402780 and CN-A 104557626 disclose a two-stage process in which in a first stage a Friedel-Crafts acylation reaction is carried out to produce 4,4′-dichlorodiphenyl sulfoxide and in a second stage the 4,4′-dichlorodiphenyl sulfoxide is oxidized to obtain DCDPS using hydrogen peroxide as oxidizing agent. The oxidation reaction thereby is carried out in the presence of acetic acid. Such a process in which 4,4′-dichlorodiphenyl sulfoxide is produced in a first stage and DCDPS is obtained in a second stage using hydrogen peroxide in excess and acetic acid as solvent also is described in SU-A 765262.

Further processes for obtaining DCDPS by reacting monochlorobenzene and thionyl chloride in a Friedel-Crafts reaction in a first stage to obtain 4,4′-dichlorodiphenyl sulfoxide and to oxidize the 4,4′-dichlorodiphenyl sulfoxide in a second stage using hydrogen peroxide as oxidizing agent and dichloromethane or dichloropropane as solvent are disclosed in CN-A 102351756 and CN-A 102351757.

A process for producing an organic sulfone by oxidation of the respective sulfoxide in the presence of at least one peroxide is disclosed in WO-A 2018/007481. The reaction thereby is carried out in a carboxylic acid as solvent, the carboxylic acid being liquid at 40° C. and having a miscibility gap with water at 40° C. and atmospheric pressure.

In all of these processes byproducts are produced and reaction aids are added which have to be removed after completing the reaction. Usually, these reaction aids are disposed.

It is an object of the present invention to provide a process for producing DCDPS which is environmentally sustainable. It is another object of the present invention to provide a process for the production of highly pure DCDPS. It was also an object of the present invention to provide a process for the production of DCDPS which is essentially colorless.

This object is achieved by a process for producing 4,4′-dichlorodiphenyl sulfone, comprising:
(a) reacting 4,4′-dichlorodiphenyl sulfoxide and an oxidizing agent in at least one carboxylic acid as solvent to obtain a reaction mixture comprising 4,4′-dichlorodiphenyl sulfone and the at least one carboxylic acid;
(b) separating the reaction mixture into a first stream comprising 4,4′-dichlorodiphenyl sulfone and a second stream comprising the at least one carboxylic acid;
(c) purifying the second stream comprising the at least one carboxylic acid by
   distilling a part of the second stream comprising the at least one carboxylic acid;
   stripping low boilers from at least a part of the second stream comprising the at least one carboxylic acid;
(d) recycling the purified at least one carboxylic acid into the reaction (a).

This process allows reusing most of the at least one carboxylic acid (in the following also termed as “carboxylic acid”) used as solvent for the reaction and thus to reduce the amount of byproducts which have to be removed and disposed.

To avoid an increase in the amount of impurities contained in the carboxylic acid, the second stream comprising the carboxylic acid which is separated off the reaction mixture is subjected to purifying (c). By purifying (c) a large amount of impurities can be removed from the carboxylic acid and thus from the process. Thereby, it is avoided that the produced DCDPS is contaminated with these impurities.

For purifying the second stream comprising the carboxylic acid 2 to 25 vol % of the second stream comprising the carboxylic acid are subjected to distillation. By distillation, low boiling impurities and high boiling impurities are removed from this part of the second stream comprising the carboxylic acid. To further remove low boilers from the second stream comprising the carboxylic acid, at least a part of this second stream is stripped with an inert gas. Thereby it is possible, to carry out the distillation of a part of the second stream before or after stripping of the whole second stream. Further, it is possible to separate the second stream into two parts and subject one part to distillation and the other part to stripping.

Therefore, in a first alternative, purifying the second stream comprising the carboxylic acid comprises:
(a1) stripping low boilers from the second stream comprising the carboxylic acid in a stripping column using a stripping gas to obtain a crude carboxylic acid;
(b1) separating the crude carboxylic acid into a first carboxylic acid stream and a second carboxylic acid stream;
(c1) distilling the second carboxylic acid stream to obtain a bottom stream comprising high boiling impurities, a top stream comprising low boiling impurities and a side stream comprising the carboxylic acid; and
(d1) mixing the first carboxylic acid stream and the side stream to obtain the purified carboxylic acid.

In a second alternative, purifying the second stream comprising the carboxylic acid comprises:
(a2) separating the second stream comprising the carboxylic acid into a first part and a second part;
(b2) stripping low boilers from the first part comprising the carboxylic acid in a stripping column using a stripping gas to obtain a crude carboxylic acid;
(c2) distilling the second part to obtain a bottom stream comprising high boiling impurities, a top stream comprising low boiling impurities and a side stream comprising the carboxylic acid; and
(d2) mixing the crude carboxylic acid obtained in (b2) and the side stream comprising the carboxylic acid obtained in (c2) to obtain the purified carboxylic acid.

And in a third alternative, purifying the second stream comprising the carboxylic acid comprises:
(a3) separating the second stream comprising the carboxylic acid into a first part and a second part;
(b3) distilling the second part to obtain a bottom stream comprising high boiling impurities, a top stream comprising low boiling impurities and a side stream comprising the carboxylic acid; and
(c3) mixing the side stream comprising the carboxylic acid obtained in (b3) and the first part of the second stream comprising the carboxylic acid to obtain crude carboxylic acid;
(d3) stripping low boilers from the crude carboxylic acid obtained in (c3) in a stripping column using a stripping gas to obtain the purified carboxylic acid.

By each of these alternatives, in which only a part of the second stream comprising the carboxylic acid is distilled and at least a part of the second stream comprising the carboxylic acid is stripped, a purified carboxylic acid is achieved which can be reused in the reaction (a).

By purifying the second stream by one of these alternatives the amount of DCDPS and carboxylic acid which is lost is much smaller than in a process where the whole second stream comprising the at least one carboxylic acid is subject to a distillation or to a distillation and stripping and the amount of DCDPS which is lost can be kept below 3 wt %, more preferred below 2 wt % and particularly below 1 wt %, based on the total amount of DCDPS produced in the reaction (a) and the amount of carboxylic acid which is lost can be kept below 3 wt %, more preferred below 2 wt % and particularly below 1 wt %, based on the total amount of carboxylic acid used in the reaction (a). A further advantage of distilling only a part of the second stream is that the energy consumption for purifying can be considerably reduced.

The reaction of 4,4′-dichlorodiphenyl sulfoxide and the oxidizing agent in at least one carboxylic acid as solvent can be operated as known by a skilled person.

Preferably, the reaction comprises producing DCDPS by reacting a solution comprising 4,4′-dichlorodiphenyl sulfoxide and the carboxylic acid, particularly a C₆-C₁₀ carboxylic acid, as organic solvent with an oxidizing agent to obtain a crude reaction product comprising 4,4′-dichlorodiphenyl sulfone, wherein the concentration of water in the reaction mixture is kept below 5 wt %.

By keeping the concentration of water below 5 wt % it is possible to use the linear C₆-C₁₀ carboxylic acid which is only slightly health hazardous and which has a good biodegradability.

Another advantage of using the linear C₆-C₁₀ carboxylic acid is that the linear C₆-C₁₀ carboxylic acid shows a good separability from water at low temperatures which allows separation of the linear C₆-C₁₀ carboxylic acid without damaging the product and which further allows recycling the linear C₆-C₁₀ carboxylic acid as solvent into the oxidation process.

In the process for producing DCDPS a solution comprising 4,4′-dichlorodiphenyl sulfoxide (in the following termed as DCDPSO) and the carboxylic acid is provided. In this solution, the carboxylic acid serves as solvent. Preferably, the ratio of DCDPSO to carboxylic acid is in a range from 1:2 to 1:6, particularly in a range from 1:2.5 to 1:3.5. Such a ratio of DCDPSO to carboxylic acid is usually sufficient to completely solve the DCDPSO in the carboxylic acid at the reaction temperature and to achieve an almost full conversion of the DCDPSO forming DCDPS and further to use as little carboxylic acid as possible. The solution comprising DCDPSO and C₆-C₁₀ carboxylic acid preferably is heated to a temperature in the range from 70 to 110° C., more preferred to a temperature in the range from 80 to 100° C. and particularly in the range from 85 to 95° C., for example 86, 87, 88, 89, 90, 91, 92, 93, 94° C., before adding the oxidizing agent.

To provide the solution, it is possible to feed DCDPSO and the carboxylic acid separately into a reactor and to mix the DCDPSO and the carboxylic acid in the reactor. Alternatively, it is also possible to mix the DCDPSO and the carboxylic acid in a separate mixing unit to obtain the solution and to feed the solution into the reactor. In a further alternative, DCDPSO and a part of the carboxylic acid are fed into the reactor as a mixture and the rest of the carboxylic acid is fed directly into the reactor and the solution is obtained by mixing the mixture of DCDPSO and part of the carboxylic acid and the rest of the carboxylic acid in the reactor.

The at least one carboxylic acid used in the reaction can be only one carboxylic acid or a mixture of at least two different carboxylic acids. Preferably the carboxylic acid is at least one aliphatic carboxylic acid. The at least one aliphatic carboxylic acid may be at least one linear or at least one branched aliphatic carboxylic acid or it may be a mixture of one or more linear and one or more branched aliphatic carboxylic acids. Preferably the aliphatic carboxylic acid is a C₆ to C₁₀ carboxylic acid, whereby it is particularly preferred that the at least one carboxylic acid is an aliphatic monocarboxylic acid. Thus, the at least one carboxylic acid may be hexanoic acid, heptanoic acid, octanoic acid nonanoic acid or decanoic acid or a mixture of one or more of said acids. For instance, the at least one carboxylic acid may be n-hexanoic acid, 2-methyl-pentanoic acid, 3-methyl-pentanoic acid, 4-methyl-pentanoic acid, n-heptanoic acid, 2-methyl-hexanoic acid, 3-methyl-hexanoic acid, 4-methyl-hexanoic acid, 5-methyl-hexanoic acid, 2-ethyl-pentanoic acid, 3-ethyl-pentanoic acid, n-octanoic acid, 2-methyl-heptanoic acid, 3-methyl-heptanoic acid, 4-methyl-heptanoic acid, 5-methyl-heptanoic acid, 6-methyl-heptanoic acid, 2-ethyl-hexanoic acid, 4-ethyl-hexanoic acid, 2-propyl pentanoic acid, 2,5-dimethylhexanoic acid, 5,5-dimethyl-hexanoic acid, n-nonanoic acid, 2-ethyl-heptanoic acid, n-decanoic acid, 2-ethyl-octanoic acid, 3-ethyl-ocantoic acid, 4-ethyl-octanoic acid. The carboxylic acid may also be a mixture of different structural isomers of one of said acids. For instance, the at least one carboxylic acid may be isononanoic acid comprising a mixture of 3,3,5-trimethyl-hexanoic acid, 2,5,5-trimethyl-hexanoic acid and 7-methyl-octanoic acid or neodecanoic acid comprising a mixture of 7,7-dimethyloctanoic acid, 2,2,3,5-tetramethyl-hexanoic acid, 2,4-dimethyl-2-isopropyl-pentanoic acid and 2,5-dimethyl-2-ethylhexanoic acid. Particularly preferably, however, the carboxylic acid is a linear C₆-C₁₀ carboxylic acid and particularly n-hexanoic acid or n-heptanoic acid.

Heating of the solution comprising DCDPSO and the carboxylic acid can be carried out in the reactor in which the reaction for obtaining the crude reaction product takes place or in any other apparatus before being fed into the reactor. Particularly preferably, the solution comprising DCDPSO and the carboxylic acid is heated to the respective temperature before being fed into the reactor. Heating of the solution for example can be carried out in a heat exchanger through which the solution flows before being fed into the reactor or more preferred in a buffer container in which the solution is stored before being fed into the reactor. If such a buffer container is used, the buffer container also may serve as mixing unit for mixing the DCDPSO and the carboxylic acid to obtain the solution.

A heat exchanger for example can be used when the process is operated continuously. Heating of the solution in a buffer container can be carried out in a continuously operated process as well as in a batchwise operated process. If a heat exchanger is used for heating the solution, any suitable heat exchanger can be used, for example a shell and tube heat exchanger, a plate heat exchanger, a spiral tube heat exchanger, or any other heat exchanger known to a skilled person. The heat exchanger thereby can be operated in counter current flow, co-current flow or cross flow.

Besides heating by using a heating fluid which usually is used in a heat exchanger or for heating in a double jacket or heating coil, also electrical heating or induction heating can be used for heating the solution.

If the solution is heated in the buffer container, any suitable container which allows heating of the contents in the container can be used. Suitable containers for example are equipped with a double jacket or a heating coil. If the buffer container additionally is used for mixing the DCDPSO and the carboxylic acid, the buffer container further comprises a mixing unit, for example a stirrer.

For carrying out the reaction, the solution preferably is provided in a reactor. This reactor can be any reactor which allows mixing and reacting of the components fed into the reactor. A suitable reactor for example is a stirred tank reactor or a reactor with forced circulation, particularly a reactor with external circulation and a nozzle to feed the circulating liquid. If a stirred tank reactor is used, any stirrer can be used. Suitable stirrers for example are axially conveying stirrers like oblique blade agitators or cross-arm stirrers or radially conveying agitators like flat blade agitators. The stirrer may have at least 2 blades, more preferred at least 4 blades. Particularly preferred is a stirrer having 4 to 8 blades, for example 6 blades. For reasons of process stability and process reliability, it is preferred that the reactor is a stirred tank reactor with an axially conveying stirrer.

For controlling the temperature in the reactor, it is further preferred to use a reactor with heat exchange equipment, for example a double jacket or a heating coil. This allows additional heating or heat dissipation during the reaction and keep the temperature constant or in a predefined temperature range at which the reaction is carried out. Preferably, the reaction temperature is kept in a range from 70 to 110° C., more preferred from 80 to 100° C. and particularly in a range from 85 to 95° C., for example 86, 87, 88, 89 90, 91, 92, 93, 94° C.

To obtain DCDPS, the solution comprising DCDPSO and carboxylic acid is oxidized by an oxidizing agent. Therefore, the oxidizing agent preferably is added to the solution to obtain a reaction mixture. From the reaction mixture the crude reaction product comprising DCDPS can be obtained.

The oxidizing agent used for oxidizing DCDPSO for obtaining DCDPS preferably is at least one peroxide. The at least one peroxide may be at least one peracid, for example one or a mixture of two or more, such as three or more peracids. Preferably, the process disclosed herein is carried out in the presence of one or two, particularly in the presence of one peracid. The at least one peracid may be a C₁ to C₁₀ peracid, which may be unsubstituted or substituted, e.g. by linear or branched C₁ to C₅ alkyl or halogen, such as fluorine. Examples thereof are peracetic acid, performic acid, perpropionic acid, percaprionic acid, pervaleric acid or pertrifluoroacetic acid. Particularly preferably the at least one peracid is a C₆ to C₁₀ peracid, for example 2-ethyl-hexanoic peracid. If the at least one peracid is soluble in water, it is advantageous to add the at least one peracid as aqueous solution. Further, if the at least one peracid is not sufficiently soluble in water, it is advantageous that the at least one peracid is dissolved in the respective carboxylic acid. Most preferably, the at least one peracid is a linear C₆ to C₁₀ peracid which is generated in situ.

Particularly preferably, the peracid is generated in situ by using hydrogen peroxide (H₂O₂) as oxidizing agent. At least a part of the added H₂O₂ reacts with the carboxylic acid forming the peracid. The H₂O₂ preferably is added as an aqueous solution, for instance of 1 to 90 wt % solution, such as a 20, 30, 40, 50, 60 or 70 wt % solution, preferably as 30 to 85 wt % solution, particularly as a 50 to 85 wt % solution, each being based on the total amount of the aqueous solution. Using a highly concentrated aqueous solution of H₂O₂, particularly a solution of 50 to 85 wt %, for example of 70 wt %, based on the total amount of the aqueous solution, may lead to a reduction of reaction time. It may also facilitate recycling of the at least one carboxylic acid.

Particularly preferably, the at least one peracid is a linear C₆ or C₇ peracid which is generated in situ. To additionally reduce the reaction time and to add only a small amount of water to the reaction mixture, it is particularly preferred that the C₆-C₁₀ carboxylic acid is n-hexanoic acid or n-heptanoic acid and the hydrogen peroxide is a 50 to 85 wt % solution.

To avoid accumulation of the oxidizing agent and to achieve a constant oxidation of the DCDPSO, it is preferred to add the oxidizing agent continuously with a feed rate from 0.002 to 0.01 mol per mol DCDPSO and minute. More preferred, the oxidizing agent is added with a feed rate from 0.003 to 0.008 mol per mol DCDPSO and minute and particularly with a feed rate from 0.004 to 0.007 mol per mol DCDPSO and minute.

The oxidizing agent can be added with a constant feed rate or with a varying feed rate. If the oxidizing agent is added with a varying feed rate, it is for example possible to reduce the feed rate with proceeding reaction within the above described range. Further it is possible to add the oxidizing agent in several steps with a stop of adding oxidizing agent between the steps. In each step during adding the oxidizing agent, the oxidizing agent can be added with a constant feed rate or a varying feed rate. Besides a decreasing feed rate with proceeding reaction, it is also possible to increase the feed rate or to switch between increasing and decreasing feed rates. If the feed rate is increased or decreased, the change in feed rate can be continuously or stepwise. Particularly preferably, the oxidizing agent is added in at least two steps wherein the feed rate in each step is constant.

If the oxidizing agent is fed in at least two steps, it is preferred to add the oxidizing agent in two steps, wherein adding the oxidizing agent to the solution preferably comprises:
(A) adding 0.9 to 1.05 mol oxidizing agent per mol 4,4′-dichlorodiphenyl sulfoxide uniformly distributed to the solution at a temperature in the range from 70 to 110° C. over a period from 1.5 to 5 h in a first step to obtain a reaction mixture;
(B) agitating the reaction mixture after completion of the first step at the temperature of the first step for 5 to 30 min without adding oxidizing agent;
(C) adding 0.05 to 0.2 mol oxidizing agent per mol 4,4′-dichlorodiphenyl sulfoxide to the reaction mixture at a temperature in the range from 80 to 110° C. over a period of less than 40 min in a second step;
(D) agitating the reaction mixture after completion of the second step at the temperature of the second step for 10 to 30 min without adding oxidizing agent,
(E) heating the reaction mixture to a temperature in the range from 95 to 110° C. and hold this temperature for 10 to 90 min to obtain a crude reaction product comprising 4,4′-dichlorodiphenyl sulfone.

If the oxidation of DCDPSO is carried out in at least two steps, for converting the DCDPSO into DCDPS, the DCDPSO is oxidized by adding the oxidizing agent in the first and second steps to the solution comprising DCDPSO and carboxylic acid.

In the first step 0.9 to 1.05 mol oxidizing agent per mol 4,4′-dichlorodiphenyl sulfoxide are added uniformly distributed to the solution at a temperature in the range from 70 to 110° C. over a period from 1.5 to 5 h. By adding the oxidizing agent over such a period an accumulation of the oxidizing agent can be avoided.

“Uniformly distributed” in this context means that the oxidizing agent can be added either continuously at a constant feed rate or at periodically changing feed rates. Besides continuous periodically changing feed rates, periodically changing feed rates also comprise discontinuously changing periodical feed rates for example feed rates where oxidizing agent is added for a defined time, then no oxidizing agent is added for a defined time and this adding and not adding is repeated until the complete amount of oxidizing agent for the first step is added. The period in which the oxidizing agent is added, is in a range from 1.5 to 5 h, more preferred in a range from 2 to 4 h and particularly in a range from 2.5 to 3.5 h. By adding the oxidizing agent uniformly distributed over such a period, it can be avoided that oxidizing agent accumulates in the reaction mixture which may result in an explosive mixture. Additionally, by adding the oxidizing agent over such a period, the process can be scaled up in an easy way as this allows also in an upscaled process to dissipate the heat from the process. On the other hand, by such an amount decomposition of the hydrogen peroxide is avoided and thus the amount of hydrogen peroxide used in the process can be minimized.

The temperature at which the first step is carried out is in the range from 70 to 110° C., preferably in the range from 85 to 100° C. and particularly in the range from 90 to 95° C. In this temperature range, a high reaction velocity can be achieved at high solubility of the DCDPSO in the carboxylic acid. This allows to minimize the amount of carboxylic acid and by this a controlled reaction can be achieved.

After the addition of the oxidizing agent in the first step is completed, the reaction mixture is agitated at the temperature of the first step for 5 to 30 min without adding oxidizing agent. By agitating the reaction mixture after completion of adding the oxidizing agent, oxidizing agent and DCDPSO which did not yet react are brought into contact to continue the reaction forming DCDPS for reducing the amount of DCDPSO remaining as impurity in the reaction mixture.

To further reduce the amount of DCDPSO in the reaction mixture, after completing of agitating without adding oxidizing agent, 0.05 to 0.2 mol oxidizing agent per DCDPSO, preferably 0.06 to 0.15 mol oxidizing agent per mol DCDPSO, and particularly 0.08 to 0.1 mol oxidizing agent per mol DCDPSO are added to the reaction mixture in the second step.

In the second step, the oxidizing agent preferably is added in a period from 1 to 40 min, more preferred in a period from 5 to 25 min and particularly in a period from 8 to 15 min. The addition of the oxidizing agent in the second step may take place in the same way as in the first step. Further, it is also possible to add the entire oxidizing agent of the second step at once.

The temperature of the second step is in the range from 80 to 110° C., more preferred in the range from 85 to 100° C. and particularly in the range from 93 to 98° C. It further is preferred that the temperature in the second step is from 3 to 10° C. higher than the temperature in the first step. More preferred the temperature in the second step is 4 to 8° C. higher than the temperature in the first step and particularly preferably, the temperature in the second step is 5 to 7° C. higher than the temperature in the first step. By the higher temperature in the second step, it is possible to achieve a higher reaction velocity.

After addition of the oxidizing agent in the second step, the reaction mixture is agitated at the temperature of the second step for 10 to 20 min to continue the oxidation reaction of DCDPSO forming DCDPS.

To complete the oxidation reaction, after agitating at the temperature of the second step without adding oxidizing agent, the reaction mixture is heated to a temperature in the range from 95 to 110° C., more preferred in the range from 95 to 105° C. and particularly in the range from 98 to 103° C. and held at this temperature for 10 to 90 min, more preferred from 10 to 60 min and particularly from 10 to 30 min.

In the oxidizing process, particularly when using H₂O₂ as oxidizing agent, water is formed. Further, water may be added with the oxidizing agent. Preferably, the concentration of the water in the reaction mixture is kept below 5 wt %, more preferred below 3 wt % and particularly below 2 wt %. By using aqueous hydrogen peroxide with a concentration of 70 to 85 wt % the concentration of water during the oxidization reaction is kept low. It even may be possible to keep the concentration of water in the reaction mixture during the oxidization reaction below 5 wt % without removing water by using aqueous hydrogen peroxide with a concentration of 70 to 85 wt %.

Additionally or alternatively, it may be necessary to remove water from the process for keeping the concentration of water in the reaction mixture below 5 wt %. To remove the water from the process, it is for example possible to strip water from the reaction mixture. Stripping thereby preferably is carried out by using an inert gas as stripping medium. If the concentration of water in the reaction mixture remains below 5 wt % when using aqueous hydrogen peroxide with a concentration of 70 to 85 wt % it is not necessary to additionally strip water. However, even in this case it is possible to strip water to further reduce the concentration.

Suitable inert gases which can be used for stripping the water are non-oxidizing gases and are preferably nitrogen, carbon dioxide, noble gases like argon or any mixture of these gases. Particularly preferably, the inert gas is nitrogen.

The amount of inert gas used for stripping the water preferably is in the range from 0 to 2 Nm³/h/kg, more preferably in the range from 0.2 to 1.5 Nm³/h/kg and particularly in the range from 0.3 to 1 Nm³/h/kg. The gas rate in Nm³/h/kg can be determined according to DIN 1343, January 1990 as relative gas flow. Stripping of water with the inert gas may take place during the whole process or during at least one part of the process. If water is stripped at more than one part of the process, between the parts stripping of water is interrupted. The interruption of stripping water is independent of the mode in which the oxidizing agent is added. For example, it is possible to add the oxidizing agent without any interruption and to strip the water with interruptions or to add the oxidizing agent in at least two steps and to strip the water continuously. Further it is also possible, to strip water only during the addition of oxidizing agent. Particularly preferably, the water is stripped by continuously bubbling an inert gas into the reaction mixture.

To avoid the formation of areas with different compositions in the reactor which may lead to different conversion rates of DCDPSO and thus to different yield and amounts of impurities, it is preferred to homogenize the reaction mixture during the first step and the second step. Homogenization of the reaction mixture can be performed by any method known to a skilled person, for example by agitating the reaction mixture. To agitate the reaction mixture, it is preferred to stir the reaction mixture. For stirring, any suitable stirrer can be used. Suitable stirrers for example are axially conveying stirrers like oblique blade agitators or cross-arm stirrers or radially conveying agitators like flat blade agitators. The stirrer may have at least 2 blades, more preferred at least 4 blades. Particularly preferred is a stirrer having 4 to 8 blades, for example 6 blades. For reasons of process stability and process reliability, it is preferred that the reactor is a stirred tank reactor with an axially conveying stirrer.

The temperature of the reaction mixture during the process can be set for example by providing a pipe inside the reactor through which a tempering medium can flow. Under the aspect of ease of reactor maintenance and/or uniformity of heating, preferably, the reactor comprises a double jacket through which the tempering medium can flow. Besides the pipe inside the reactor or the double jacket the tempering of the reactor can be performed in each manner known to a skilled person, for example by withdrawing a stream of the reaction mixture from the reactor, passing the stream through a heat exchanger in which the stream is tempered and recycle the tempered stream back into the reactor.

To support the oxidation reaction, it is further advantageous to additionally add at least one acidic catalyst to the reaction mixture. The acidic catalyst may be at least one, such as one or more, such as a mixture of two or three additional acids. An additional acid in this context is an acid which is not the carboxylic acid which serves as solvent. The additional acid may be an inorganic or organic acid, with the additional acid preferably being an at least one strong acid. Preferably, the strong acid has a pKₐ value from −9 to 3, for instance −7 to 3 in water. A person skilled in the art appreciates that such acid dissociation constant values, Kₐ, can be for instance found in a compilation such as in IUPAC, Compendium of Chemical Terminology, 2^{nd} ed. “Gold Book”, Version 2.3.3, 2014 Feb. 24, page 23. The person skilled in the art appreciates that such pKₐ values relate to the negative logarithm value of the Kₐ value. it is more preferred that the at least one strong acid has a negative pKₐ value, such as from −9 to −1 or −7 to −1 in water.

Examples for inorganic acids being the at least one strong acid are nitric acid, hydrochloric acid, hydrobromic acid, perchloric acid, and/or sulfuric acid. Particularly preferably, one strong inorganic acid is used, in particular sulfuric acid. While it may be possible to use the at least one strong inorganic acid as aqueous solution, it is preferred that the at least one inorganic acid is used neat. Suitable strong organic acids for example are organic sulfonic acids, whereby it is possible that at least one aliphatic or at least one aromatic sulfonic acid or a mixture thereof is used. Examples for the at least one strong organic acid are para-toluene sulfonic acid, methane sulfonic acid or trifluormethane sulfonic acid. Particularly preferably the strong organic acid is methane sulfonic acid. Besides using either at least one inorganic strong acid or at least one organic strong acid, it is also possible to use a mixture of at least one inorganic strong acid and at least one organic strong acid as acidic catalyst. Such a mixture for example may comprise sulfuric acid and methane sulfonic acid.

The acidic catalyst preferably is added in catalytic amounts. Thus, the amount of acidic catalyst used may be in the range from 0.1 to 0.3 mol per mol DCDPSO, more preferred in the range from 0.15 to 0.25 mol per mol DCDPSO. However, it is preferred to employ the acidic catalyst in an amount of less than 0.1 mol per mol DCDPSO, such as in an amount from 0.001 to 0.08 mol per mol DCDPSO, for example from 0.001 to 0.03 mol per mol DCDPSO. Particularly preferably, the acidic catalyst is used in an amount from 0.005 to 0.01 mol per mol DCDPSO.

The oxidization reaction for obtaining DCDPS can be carried out as a batch process, as a semi continuous process or as a continuous process. Preferably, the oxidization reaction is carried out batchwise. The oxidation reaction can be carried out at atmospheric pressure or at a pressure which is below or above atmospheric pressure, for example in a range from 10 to 900 mbar(abs). Preferably, the oxidation reaction is carried out at a pressure in a range from 200 to 800 mbar(abs) and particularly in a range from 400 to 700 mbar(abs).

The oxidization reaction can be carried out under ambient atmosphere or inert atmosphere. If the oxidization reaction is carried out under inert atmosphere, it is preferred to purge the reactor with an inert gas before feeding the DCDPSO and the carboxylic acid. If the oxidization reaction is carried out under an inert atmosphere and the water formed during the oxidation reaction is stripped with an inert gas, it is further preferred that the inert gas used for providing the inert atmosphere and the inert gas which is used for stripping the water is the same. It is a further advantage of using an inert atmosphere that the partial pressure of the components in the oxidization reaction, particularly the partial pressure of water is reduced.

To obtain the DCDPS as product, the reaction mixture is separated into a first stream comprising DCDPS and a second stream comprising the carboxylic acid.

Preferably, for separating the reaction mixture into the first stream comprising DCDPS and the second stream comprising the carboxylic acid, the reaction mixture is cooled to a temperature below the saturation point of DCDPS to obtain a suspension comprising crystallized DCDPS and a liquid phase and the suspension is separated by a solid-liquid separation into residual moisture comprising DCDPS and mother liquor. The solid-liquid separation thereby can be carried out for example by filtration or centrifugation.

The saturation point denotes the temperature of the reaction mixture at which DCDPS starts to crystallize. This temperature depends on the concentration of the DCDPS in the reaction mixture. The lower the concentration of DCDPS in the reaction mixture, the lower is the temperature at which crystallization starts.

To purify the residual moisture comprising DCDPS (in the following termed as “moist DCDPS”), the moist DCDPS is washed with an aqueous base and subsequently with water.

By washing with an aqueous base, the anions of the carboxylic acid react with the cations of the aqueous base forming an organic salt. A part of this organic salt is removed with the aqueous base during washing with the aqueous base. The rest of the organic salt remains in the moist DCDPS and is removed from the moist DCDPS by the subsequent washing with water.

To reduce the amount of carboxylic acid which is withdrawn from the process and disposed, the aqueous base preferably is mixed with a strong acid after being used for washing. By mixing the aqueous base after being used for washing with the strong acid the anion of the organic salt reacts with the cation of the strong acid and the cation of the organic salt reacts with the anion of the strong acid, whereby carboxylic acid and an inorganic salt are formed. This allows reducing the amount of carboxylic acid which is disposed, because also that part of the carboxylic acid which formed the organic salt during washing with the aqueous base has not to be disposed but can be reused after being separated off. A further advantage of adding the strong acid after washing and thus forming the carboxylic acid and the inorganic salt and reusing of the carboxylic acid is that the total organic carbon (TOC) in the aqueous phase is reduced and thus the aqueous phase is easier to dispose. Preferably, the amounts of aqueous base used for washing and strong acid added to the aqueous base after the aqueous base was used for washing are equimolar.

The cooling for crystallizing DCDPS can be carried out in any crystallization apparatus or any other apparatus which allows cooling of the reaction mixture, for example an apparatus with surfaces that can be cooled such as a vessel or tank with cooling jacket, cooling coils or cooled baffles like so-called “power baffles”.

Cooling of the reaction mixture for crystallization of the DCDPS can be performed either continuously or batchwise. To avoid precipitation and fouling on cooled surfaces, it is preferred to carry out the cooling in a gastight closed vessel by mixing the reaction mixture with water in the gastight closed vessel to obtain a liquid mixture and cooling the liquid mixture to a temperature below the saturation point of 4,4′-dichlorodiphenyl sulfone by
(i) reducing the pressure in the gastight closed vessel to a pressure at which the water starts to evaporate,
(ii) condensing the evaporated water by cooling
(iii) mixing the condensed water into the liquid mixture in the gastight closed vessel to obtain a suspension comprising crystallized 4,4′-dichlorodiphenyl sulfone;

This process allows for cooling the DCDPS comprising reaction mixture without cooling surfaces onto which particularly at starting the cooling process crystallized DCDPS accumulates and forms a solid layer. This enhances the efficiency of the cooling process. Also, additional efforts to remove this solid layer can be avoided.

If cooling is performed according to this process, the suspension which is subjected to the solid-liquid separation additionally contains water besides the crystallized DCDPS and the carboxylic acid.

Particularly when a carboxylic acid is used as solvent which has a boiling point above 150° C. at 1 bar, cooling by reducing the pressure to evaporate solvent, to condense the evaporated solvent by cooling and recycling the condensed solvent back into the gastight vessel would require a high energy consumption to achieve the necessary low pressures. Using higher temperatures to evaporate solvent for shifting the saturation point such that DCDPS crystallizes on the other hand would have a negative effect on the DCDPS; particularly a change in color of the DCDPS cannot be excluded. By mixing the reaction mixture with water and to evaporate, condense and recycle the condensed water, it is possible to shift the saturation point by cooling without evaporating solvent at high temperatures or to reduce the pressure to very low values which is very energy consuming. Surprisingly, cooling and crystallization of DCDPS by adding water, reducing the pressure to evaporate water, condensing the water by cooling and recycle the condensed water and mix it into the reaction mixture even can be carried out when carboxylic acids are used as solvent which have a poor solubility in water.

To crystallize the DCDPS, it is preferred to provide crystal nuclei. To provide the crystal nuclei, it is possible to use dried crystals which are added to the reaction mixture or to add a suspension comprising particulate DCDPS as crystal nuclei. If dried crystals are used but the crystals are too big, it is possible to grind the crystals into smaller particles which can be used as crystal nuclei. Further, it is also possible to provide the necessary crystal nuclei by applying ultrasound to the liquid mixture. Preferably, the crystal nuclei are generated in situ in an initializing step. The initializing step preferably comprises following steps before reducing pressure in step (i):
reducing the pressure in the gastight closed vessel such that the boiling point of the water in the liquid mixture is in the range from 80 to 95° C.;
evaporating water until an initial formation of solids takes place;
increasing the pressure in the vessel and heating the liquid mixture in the gastight closed vessel to a temperature in the range from 1 to 10° C. below the saturation point of DCDPS.

By reducing the pressure in the vessel such that the water starts to evaporate at a temperature in the range from 80 to 95° C., more preferred in the range from 83 to 92° C., the following evaporation of water leads to a saturated solution and the precipitation of DCDPS. By the following pressure increase and heating the liquid mixture in the gastight closed vessel to a temperature in the range from 1 to 10° C. below the saturation point of DCDPS the solidified DCDPS starts to partially dissolve again. This has the effect that the number of crystal nuclei is reduced which allows producing a smaller amount of crystals with a bigger size. Further it is ensured that an initial amount of crystal nuclei remains in the gastight closed vessel. Cooling, particularly by reducing the pressure, can be started immediately after a pre-set temperature within the above ranges is reached to avoid complete dissolving of the produced crystal nuclei. However, it is also possible to start cooling after a dwell time for example of 0.5 to 1.5 h at the pre-set temperature.

For generating the crystal nuclei in the initializing step, it is possible to only evaporate water until an initial formation of solids take place. It is also possible to entirely condense the evaporated water by cooling and to return all the condensed water into the gastight closed vessel. The latter has the effect that the liquid mixture in the gastight closed vessel is cooled and solid forms. A mixture of both approaches, where only a part of the evaporated and condensed water is returned into the gastight vessel, is also viable.

Cooling of the liquid mixture by reducing the pressure, evaporate water, condense the evaporated water by cooling and mixing the condensed water into the liquid mixture can be carried out batchwise, semi-continuously or continuously.

Particularly in a batchwise process, the pressure reduction to evaporate water and thereby to cool the liquid mixture can be for example stepwise or continuously. If the pressure reduction is stepwise, it is preferred to hold the pressure in one step until a predefined rate in temperature decrease can be observed, particularly until the predefined rate is “0” which means that no further temperature decrease occurs. After this state is achieved, the pressure is reduced to the next pressure value. In this case the steps for reducing the pressure all can be the same or can be different. If the pressure is reduced in different steps, it is preferred to reduce the size of the steps with decreasing pressure. Preferably, the steps in which the pressure is decreased are in a range from 10 to 800 mbar, more preferred in a range from 30 to 500 mbar and particularly in a range from 30 to 300 mbar.

If the pressure reduction is continuously, the pressure reduction can be for example linearly, hyperbolic, parabolic or in any other shape, wherein it is preferred for a non-linear decrease in pressure to reduce the pressure in such a way that the pressure reduction decreases with decreasing pressure. If the pressure is reduced continuously, it is preferred to reduce the pressure with a rate from 130 to 250 mbar/h, particularly with a rate from 180 to 220 mbar/h. Moreover, the pressure can be reduced bulk temperature controlled by use of a process control system (PCS), whereby a stepwise linear cooling profile is realized.

Preferably, the pressure reduction is temperature controlled with a stepwise cooling profile from 5 to 25 K/h to approximate a constant supersaturation with increasing solid content and thus, more crystalline surface for growth.

If the cooling and thereby the crystallization is carried out in a semi-continuous process, the pressure preferably is reduced stepwise, wherein the semi-continuous process for example can be realized by using at least one gastight vessel for each pressure step, respectively temperature step. For cooling the liquid mixture, the liquid mixture is fed into the first gastight vessel having the highest temperature and cooled to a first temperature. Then the liquid mixture is withdrawn from the first gastight vessel and fed into a second gastight vessel having a lower pressure. This process is repeated until the liquid mixture is fed into the gastight vessel having the lowest pressure. As soon as the liquid mixture is withdrawn from one vessel, fresh liquid mixture can be fed into that vessel, wherein the pressure in the vessel preferably is kept constant. “Constant” in this context means that variations in pressure which depend on withdrawing and feeding liquid mixture into the respective tank are kept as low as technically possible but cannot be excluded.

Besides carrying out the process batchwise or semi-continuous, it is also possible to perform the process continuously. If the cooling and thus the crystallization of DCDPS is performed continuously, it is preferred to operate the cooling and crystallization stepwise in at least two steps, particularly in two to three steps, wherein for each step at least on gastight closed vessel is used. If the cooling and crystallization is carried out in two steps, in a first step the liquid mixture preferably is cooled to a temperature in the range from 40 to 90° C. and in a second step preferably to a temperature in the range from −10 to 50° C. If the cooling is operated in more than two steps, the first step preferably is operated at a temperature in the range from 40 to 90° C. and the last step at a temperature in the range from −10 to 30° C. The additional steps are operated at temperatures between these ranges with decreasing temperature from step to step. If the cooling and crystallization is performed in three steps, the second step for example is operated at a temperature in the range from 10 to 50° C.

If the cooling and crystallization is carried out continuously, a stream of the suspension is continuously withdrawn from the last gastight vessel. The suspension then is fed into the solid-liquid-separation (b). To keep the liquid level in the gastight closed vessels within predefined limits fresh liquid mixture comprising DCDPS, carboxylic acid and water can be fed into each gastight closed vessel in an amount corresponding or essentially corresponding to the amount of suspension withdrawn from the respective gastight closed vessel. The fresh liquid mixture either can be added continuously or batchwise each time a minimum liquid level in the gastight closed vessel is reached.

Independently of being carried out batchwise or continuously, crystallization preferably is continued until the solids content in the suspension in the last step of the crystallization is in the range from 5 to 50 wt %, more preferred in the range from 5 to 40 wt % and particularly in the range from 20 to 40 wt %, based on the mass of the suspension.

To achieve this solids content in the suspension, it is preferred to reduce the pressure in (i) until the suspension which is obtained by the cooling has cooled down to a temperature in the range from 10 to 30° C., preferably in the range from 15 to 30° C. and particularly in the range from 20 to 30° C.

The pressure at which this temperature is achieved depends on the amount of water in the liquid mixture. Preferably, the amount of water mixed to the reaction mixture is such that the amount of water in the liquid mixture is in the range from 10 to 60 wt % based on the total amount of the liquid mixture. More preferred, the amount of water mixed to the reaction mixture is such that the amount of water in the liquid mixture is in the range from 10 to 50 wt % based on the total amount of the liquid mixture and, particularly, the amount of water mixed to the reaction mixture is such that the amount of water in the liquid mixture is in the range from 15 to 35 wt % based on the total amount of the liquid mixture.

Even though the cooling and crystallization can be carried out continuously or batchwise, it is preferred to carry out the cooling and crystallization batchwise. Batchwise cooling and crystallization allows a higher flexibility in terms of operating window and crystallization conditions and is more robust against variations in process conditions.

To support cooling of the liquid mixture it is further possible to provide the gastight closed vessel with coolable surfaces for an additional cooling. The coolable surfaces for example can be a cooling jacket, cooling coils or cooled baffles like so called “power baffles”. Surprisingly, forming of precipitations and fouling on coolable surfaces can be avoided or at least considerably reduced, if the additional cooling is started not before the temperature of the liquid mixture is reduced to a temperature in the range from 20 to 60° C., more preferred in a range from 20 to 50° C. and particularly in a range from 20 to 40° C.

After completing the cooling and crystallization by pressure reduction, the process is finished and preferably the pressure is set to ambient pressure, again. After reaching ambient pressure, the suspension which formed by cooling the liquid mixture in the gastight closed vessel is subjected to the solid-liquid separation. In the solid liquid separation process, the solid DCDPS formed by cooling is separated from the carboxylic acid and the water.

Independently of whether the cooling and crystallization is performed continuously or batchwise, the solid-liquid-separation (b) can be carried out either continuously or batchwise, preferably continuously.

If the cooling and crystallization is carried out batchwise and the solid-liquid-separation is carried out continuously at least one buffer container is used into which the suspension withdrawn from the gastight closed vessel is filled. For providing the suspension a continuous stream is withdrawn from the at least one buffer container and fed into a solid-liquid-separation apparatus. The volume of the at least one buffer container preferably is such that each buffer container is not totally emptied between two filling cycles in which the contents of the gastight closed vessel is fed into the buffer container. If more than one buffer container is used, it is possible to fill one buffer container while the contents of another buffer container are withdrawn and fed into the solid-liquid-separation. In this case the at least two buffer containers are connected in parallel. The parallel connection of buffer containers further allows filling the suspension into a further buffer container after one buffer container is filled. An advantage of using at least two buffer containers is that the buffer containers may have a smaller volume than only one buffer container. This smaller volume allows a more efficient mixing of the suspension to avoid sedimentation of the crystallized DCDPS. To keep the suspension stable and to avoid sedimentation of solid DCDPS in the buffer container, it is possible to provide the buffer container with a device for agitating the suspension, for example a stirrer, and to agitate the suspension in the buffer container. Agitating preferably is operated such that the energy input by stirring is kept on a minimal level, which is high enough to suspend the crystals but prevents them from breakage. For this purpose, the energy input preferably is preferably in the range from 0.2 to 0.5 W/kg, particularly in the range from 0.25 to 0.4 W/kg.

If the cooling and crystallization and the solid-liquid-separation are carried out batchwise the contents of the gastight closed vessel directly can be fed into a solid-liquid-separation apparatus as long as the solid-liquid separation apparatus is large enough to take up the whole contents of the gastight closed vessel. In this case it is possible to omit the buffer container. It is also possible to omit the buffer container when cooling and crystallization and the solid-liquid-separation are carried out continuously. In this case also the suspension directly is fed into the solid-liquid-separation apparatus. If the solid-liquid separation apparatus is too small to take up the whole contents of the gastight closed vessel, also for batchwise operation at least one additional buffer container is necessary to allow to empty the gastight closed vessel and to start a new batch.

If the cooling and crystallization are carried out continuously and the solid-liquid-separation is carried out batchwise the suspension withdrawn from the gastight closed vessel is fed into the buffer container and each batch for the solid-liquid-separation is withdrawn from the buffer container and fed into the solid-liquid-separation apparatus.

The solid-liquid-separation for example comprises a filtration, centrifugation or sedimentation. Preferably, the solid-liquid-separation is a filtration. In the solid-liquid-separation liquid mother liquor comprising carboxylic acid and water is removed from the solid DCDPS and residual moisture containing DCDPS (in the following also termed as “moist DCDPS”) is obtained as product. If the solid-liquid-separation is a filtration, the moist DCDPS is called “filter cake”.

Independently of carried out continuously or batchwise, the solid-liquid-separation preferably is performed at ambient temperature or temperatures below ambient temperature, preferably at ambient temperature. It is possible to feed the suspension into the solid-liquid-separation apparatus with elevated pressure for example by using a pump or by using an inert gas having a higher pressure, for example nitrogen. If the solid-liquid-separation is a filtration and the suspension is fed into the filtration apparatus with elevated pressure the differential pressure necessary for the filtration process is realized by setting ambient pressure to the filtrate side in the filtration apparatus. If the suspension is fed into the filtration apparatus at ambient pressure, a reduced pressure is set to the filtrate side of the filtration apparatus to achieve the necessary differential pressure. Further, it is also possible to set a pressure above ambient pressure on the feed side of the filtration apparatus and a pressure below ambient pressure on the filtrate side or a pressure below ambient pressure on both sides of the filter in the filtration apparatus, wherein also in this case the pressure on the filtrate side must be lower than on the feed side. Further, it is also possible to operate the filtration by only using the static pressure of the liquid layer on the filter for the filtration process. Preferably, the pressure difference between feed side and filtrate side and thus the differential pressure in the filtration apparatus is in the range from 100 to 6000 mbar(abs), more preferred in the range from 300 to 2000 mbar(abs) and particularly in the range from 400 to 1500 mbar(abs), wherein the differential pressure also depends on the filters used in the solid-liquid-separation (b).

To carry out the solid-liquid-separation (b) any solid-liquid-separation apparatus known by the skilled person can be used. Suitable solid-liquid-separation apparatus are for example an agitated pressure nutsche, a rotary pressure filter, a drum filter, a belt filter or a centrifuge. The pore size of the filters used in the solid-liquid-separation apparatus preferably is in the range from 1 to 1000 μm, more preferred in the range from 10 to 500 μm and particularly in the range from 20 to 200 μm.

The apparatus for solid-liquid separation, particularly the filtration apparatus, preferably is made of a nickel-base alloy or stainless steel. Further it is also possible to use coated steel, wherein the coating is made of a material which is resistant against corrosion. If the solid-liquid-separation is a filtration, the filtration apparatus preferably comprises a filter element which is made of a material which has a good or very good chemical resistance. Such materials can be polymeric materials or chemical resistant metals as described above for the used apparatus. Filter elements for example can be filter cartridges, filter membranes, or filter cloth. If the filter element is a filter cloth, preferred materials additionally are flexible, particularly flexible polymeric materials such as those which can be fabricated into wovens. These can for instance be polymers which can be drawn or spun into fibers. Particularly preferred as material for the filter element are polyether ether ketone (PEEK), polyamide (PA) or fluorinated polyalkylenes, for example ethylene chlorotrifluoroethylene (ECTFE), polytetrafluoroethylene (PTFE), polyvinylidene fluoride (PVDF), fluorinated ethylene-propylene (FEP).

Particularly preferably, cooling and crystallization is carried out batchwise and the solid-liquid-separation is operated continuously.

If the solid-liquid-separation is a filtration, it is possible to carry out the following washing of the filter cake in the filtration apparatus, independently of whether the filtration is operated continuously or batchwise. After washing, the filter cake is removed as product.

In a continuous solid-liquid-separation process, the moist DCDPS can be removed continuously from the solid-liquid-separation apparatus and afterwards the washing of the moist DCDPS takes place. In the case the solid-liquid separation is a filtration and a continuous belt filter is used, it is preferred to filtrate the suspension, to transport the thus originating filter cake on the filter belt and to wash the filter cake at a different position in the same filtration apparatus. However, if the solid-liquid separation is a continuously operated filtration, it is preferred to carry out the solid-liquid-separation and the subsequent washing in the same apparatus.

If the solid-liquid separation is a filtration process, it is further also possible to operate the filtration semi-continuously. In this case the suspension is fed continuously into the filtration apparatus and the filtration is performed for a specified process time. Afterwards the filter cake produced during the filtration is washed in the same filtration apparatus. The process time for performing the filtration for example may depend on the differential pressure. Due to the increasing filter cake the differential pressure in the filtration apparatus increases. To determine the process time for the filtration, it is for example possible to define a target differential pressure up to which the filtration is carried out in a first filtration apparatus. Thereafter the suspension is fed into a second or further filtration apparatus in which filtration is continued. This allows to continuously perform the filtration. In those apparatus where the filtration is completed, the filter cake can be washed and withdrawn after finishing the washing. If necessary, the filtration apparatus can be cleaned after the filter cake is withdrawn. After the filter cake is withdrawn and the filter apparatus is cleaned when necessary, the filtration apparatus can be used again for filtration. If the washing of the filter cake and the optional cleaning of the filtration apparatus needs more time than the time for the filtration in one filtration apparatus at least two filtration apparatus are used to allow continuous feeding of the suspension in a filtration apparatus while in the other apparatus the filter cake is washed or the filtration apparatus are cleaned.

In each filtration apparatus of the semi-continuous process, the filtration is carried out batchwise. Therefore, if the filtration and washing are carried out batchwise, the process corresponds to the process in one apparatus of the above described semi-continuous process.

After the solid-liquid separation is completed, the moist DCDPS which can be the filter cake is washed to remove remainders of the carboxylic acid and further impurities, for example undesired by-products which formed during the process for producing the DCDPS.

Washing thereby is carried out in at least two phases. In a first phase, the moist DCDPS is washed with an aqueous base which is followed by washing with water in a second phase.

To remove the remainders of the carboxylic acid from the moist DCDPS, the aqueous base used for washing in the first phase preferably is an aqueous alkali metal hydroxide, for example aqueous potassium hydroxide or sodium hydroxide, particularly sodium hydroxide. If an alkali metal hydroxide is used as aqueous base, the aqueous alkali metal hydroxide preferably comprises from 1 to 50 wt % alkali metal hydroxide based on the total amount of aqueous alkali metal hydroxide, more preferred from 1 to 20 wt % alkali metal hydroxide based on the total amount of aqueous alkali metal hydroxide and particularly from 2 to 10 wt % alkali metal hydroxide based on the total amount of aqueous alkali metal hydroxide. This amount is sufficient for properly washing the moist DCDPS.

By using the aqueous alkali metal hydroxide, the anion of the carboxylic acid reacts with the alkali metal cation of the alkali metal hydroxide forming an organic salt and water. In difference to the carboxylic acid which generally is not soluble in water and depending on the carboxylic acid also even may be immiscible with water, the organic salt formed by reaction with the aqueous base is soluble in water and thus remainders which are not removed with the aqueous alkali metal hydroxide and the water formed by the reaction can be removed from the moist DCDPS by washing with water. This allows to achieve DCDPS as product which contains less than 1 wt %, preferably less than 0.7 wt % and particularly less than 0.5 wt % organic impurities.

For obtaining DCDPS with such a small content of organic impurities, the amount of the aqueous base, particularly the alkali metal hydroxide used for the washing in the first phase preferably is in a range from 0.5 to 10 kg per kg dry DCDPS, more preferred in a range from 1 to 6 kg per kg dry DCDPS and particularly in a range from 2 to 5 kg per kg dry DCDPS.

As the water of the aqueous base and the water produced by the reaction of the anion of the base with the carboxylic acid generally is not sufficient to remove all of the organic salt and as further part of the aqueous base may stay in the moist DCDPS, the moist DCDPS is washed with water in the second phase. By washing with water, remainders of the organic salt and of the aqueous base which did not react are removed. The water then can be easily removed from the DCDPS by usual drying processes known to a skilled person to obtain dry DCDPS as product. Alternatively, it is possible to use the water wet DCDPS which is obtained after washing with water in subsequent process steps.

The amount of water used for washing in the second phase preferably is chosen such that the aqueous base remaining in the DCDPS after washing with the aqueous base is removed. This can be achieved for example by measuring the pH value of the moist DCDPS. Washing is continued until the DCDPS is neutral which means a pH value in the range from 6.5 to 7.5, preferably in the range from 6.8 to 7.2 and particularly in the range from 6.9 to 7.1. This can be achieved by using water for washing after washing with the aqueous base in an amount which preferably is in the range from 0.5 to 10 kg per kg dry DCDPS, more preferred in the range from 1 to 7 kg per kg dry DCDPS and particularly in the range from 1 to 5 kg per kg dry DCDPS. Using such an amount of water for washing in the second phase has the advantage that the amount of waste water which has to be withdrawn from the process and passed into a purification plant for cleaning can be kept on a very low level.

The washing with water in the second phase preferably is carried out in two washing steps. In this case, it is particularly preferred to use fresh water for the washing in the second washing step and to use the water which has been used in the second washing step in the first washing step. This allows the amount of water which is used for washing in total to be kept low.

If the solid-liquid-separation is a filtration, it is possible to carry out the following washing of the filter cake in the filtration apparatus, independently of whether the filtration is operated continuously or batchwise. After washing, the filter cake is removed as product.

Besides carrying out filtration and washing of the filter cake in one apparatus, it is also possible to withdraw the filter cake from the filtration apparatus and wash it in a subsequent washing apparatus. If the filtration is carried out in a belt filter, it is possible to convey the filter cake on the filter belt into the washing apparatus. For this purpose, the filter belt is designed in such a way that it leaves the filtration apparatus and enters into the washing apparatus. Besides transporting the filter cake on a filter belt from the filtration apparatus into the washing apparatus it is also possible to collect the filter cake with a suitable conveyor and feed the filter cake from the conveyor into the washing apparatus. If the filter cake is withdrawn from the filtration apparatus with a suitable conveyor the filter cake can be withdrawn from the filtration apparatus as a whole, or in smaller pieces such as chunks or pulverulent. Chunks for instance arise if the filter cake breaks when it is withdrawn from the filtration apparatus. To achieve a pulverulent form, the filter cake usually must be comminuted. Independently from the state of the filter cake, for washing the filter cake is brought into contact with the aqueous base and subsequently with water. For example, the filter cake can be put on a suitable tray in the washing apparatus and the aqueous base flows through the tray and the filter cake. Further it is also possible to break the filter cake into smaller chunks or particles and to mix the chunks or particles with the aqueous base. Subsequent the thus produced mixture of chunks or particles of the filter cake and the aqueous base is filtrated to remove the aqueous base. If the washing is carried out in a separate washing apparatus, the washing apparatus can be any suitable apparatus. Preferably the washing apparatus is a filter apparatus which allows to use a smaller amount of aqueous base and to separate the aqueous base from the solid DCDPS in only one apparatus. However, it is also possible to use for example a stirred tank as washing apparatus. In this case it is necessary to separate the aqueous base from the washed DCDPS in a following step, for example by filtration or centrifugation. After the washing with the aqueous base, the washing with water is carried out in the same way. Thereby, for washing with the aqueous base and the washing with water only one apparatus can be used or the washing with the aqueous base and the subsequent washing with water are carried out in different apparatus.

If the solid-liquid-separation (b) is carried out by centrifugation, depending on the centrifuge it might be necessary to use a separate washing apparatus for washing the moist DCDPS. However, usually a centrifuge can be used which comprises a separation zone and a washing zone or the washing can be carried out after centrifuging in the centrifuge.

Washing of the moist DCDPS preferably is operated at ambient temperature. It is also possible to wash the moist DCDPS at temperatures different to ambient temperature, for instance above ambient temperature. If the washing is carried out in the filtration apparatus, for washing the filter cake a differential pressure must be established. This is possible for example by feeding the aqueous base in the first phase and the water in the second phase for washing the filter cake at a pressure above ambient pressure and withdraw the aqueous base and the water, respectively, after passing the filter cake at a pressure below the pressure at which the aqueous base and the water are fed, for example at ambient pressure. Further it is also possible to feed the aqueous base and the water for washing the filter cake at ambient pressure and withdraw the aqueous base and the water after passing the filter cake at a pressure below ambient pressure.

Particularly the aqueous base which was used for washing the moist DCDPS contains either carboxylic acid or the organic salt of the carboxylic acid. To reduce the amount of carboxylic acid which is withdrawn with the water and subjected to purification in a purification plant and thereby completely removed, according to the invention, the aqueous base is mixed with a strong acid after being used for washing. By mixing with the strong acid, the organic salt which formed during washing with the aqueous base reacts with the strong acid forming the carboxylic acid from the anion of the organic salt and a second salt from the anion of the strong acid. The strong acid preferably is selected such that the second salt which forms has a good solubility in water and a poor solubility in the carboxylic acid. In this context “good solubility” means at least 20 g per 100 g solvent can be dissolved and “poor solubility” means that less than 5 g per 100 g solvent can be dissolved in the solvent.

The poor solubility of the second salt in the carboxylic acid has the effect that the carboxylic acid which can be recovered comprises less than 3 ppm wt % impurities based on the total mass of the carboxylic acid. This allows further use of the carboxylic acid without further purification steps.

Depending on the aqueous base which is used for the washing of the moist DCDPS, the strong acid preferably is sulfuric acid or a sulfonic acid, like paratoluene sulfonic acid or alkane sulfonic acid, for example methane sulfonic acid. If the aqueous base is an alkali metal hydroxide, the strong acid particularly preferably is sulfuric acid. Particularly if a strong acid is used as acidic catalyst in the reaction, the strong acid which is used for mixing with the aqueous base to remove the carboxylic acid and the acid used as acidic catalyst preferably are the same.

Mixing of the aqueous base after being used for washing and the strong acid can be carried out in any mixer known to a skilled person. Suitable mixers for mixing the aqueous base after being used for washing and the strong acid for example is a static mixer, a tube, a dynamic mixer like a mixing pump, or a stirred vessel.

To allow reusing the carboxylic acid, the carboxylic acid has to be separated from the aqueous phase. This preferably is carried out by a phase separation. The carboxylic acid separated by the phase separation can be used in any process in which a respective carboxylic acid is used. However, it is particularly preferred to recycle the carboxylic acid into the reaction (a) for producing the DCDPS. If the carboxylic acid contains impurities after being separated off, it is further possible, to subject the carboxylic acid to additional purifying steps like washing or distillation to remove high boiling or low boiling impurities.

Due to the comparatively small amount of carboxylic acid in the aqueous base after being mixed with the strong acid, it is possible to add at least a part of the carboxylic acid comprising filtrate to the aqueous base mixed with the strong acid before carrying out the phase separation. This allows to improve the efficiency of the phase separation.

Particularly in case the cooling and crystallization is carried out in the gastight closed vessel by adding water and reducing the pressure, the carboxylic acid comprising filtrate additionally contains water. To allow reuse of the carboxylic acid in this case also the filtrate must be subject to a phase separation. Mixing the aqueous base mixed with the strong acid and the carboxylic acid comprising filtrate in this case has the additional advantage that only one phase separation has to be carried out for separating the organic carboxylic acid from the aqueous phase.

Depending on the amounts of organic phase and aqueous phase and the process used for phase separation, it may be necessary to increase the amount of the aqueous phase in the mixture. This can be achieved for example by circulating at least a part of the aqueous phase through the phase separation apparatus and the mixing device. Preferably, the phase separation apparatus and the mixing device are combined in one apparatus, particularly a mixer-settler and the at least part of the aqueous phase is circulated through the mixer-settler. For circulating at least a part of the aqueous phase through the phase separation apparatus and the mixing device, preferably the mixer-settler, the at least part of the aqueous phase is branched off the total aqueous phase withdrawn from the phase separation apparatus and mixed with the carboxylic acid comprising filtrate and the aqueous base mixed with the strong acid before this mixture is subjected to the phase separation again.

Mixing of the carboxylic acid comprising filtrate and the aqueous base mixed with the strong acid and—if applicable—with the part of the aqueous phase to be circulated can be carried out in a separate mixing device or preferably in the mixing part of a mixer-settler in which also the phase separation takes place. Mixing and phase separation can be carried out batchwise or continuously. If mixing and phase separation are carried out continuously and the mixture flows through the mixer settler, for mixing the several streams, preferably a coalescing aid is placed in the mixing part of the mixer-settler. Such a coalescing aid for example is a packed layer like a structured packing or a random packing. Further, a knitted mesh or a coalescer can be used as coalescing aid. Filling bodies used for the random packing can be for example Pall®-rings, Raschig®-rings or saddles.

To avoid particle clogging, after filtration the mother liquor can be used for flushing the outlet for the aqueous base of the filter.

If the phase separation is carried out batchwise, it is possible to feed all streams separately into a mixer-settler, mix them, for example by agitating like stirring, then stop stirring and let the phases separate. After phase separation is completed, the aqueous phase and the organic phase can be withdrawn from the mixer-settler separately.

Further, independently of carrying out the phase separation batchwise or continuously, it is also possible to mix the streams before feeding into a phase separation apparatus. Mixing in this case can be carried out in a static or dynamic mixer to which the streams are added or preferably by feeding all streams into one tube and mixing results from turbulence in the stream. If a static mixer is used, the mixer may contain a coalescing aid as described above.

Besides feeding the part of the aqueous phase which was branched off for circulating into the phase separation apparatus before or after mixing with the carboxylic acid comprising filtrate and the aqueous base after being mixed with the strong acid, it is also possible to recirculate the part of the aqueous phase into the mixing of the aqueous base with the strong acid.

Additionally or alternatively, it is also possible to increase the amount of the aqueous phase by feeding at least a part of the water which was used for washing in the second phase after the washing with the aqueous base, into the phase separation. By feeding at least a part of this water into the phase separation even traces of organic impurities, particularly carboxylic acid which may still be comprised in the DCDPS after washing with the aqueous base can be regained.

In an alternative, it is also possible to mix the mother liquor and the organic phase obtained in the phase separation. In this case, the mother liquor may undergo a phase separation to remove water from the mother liquor before mixing, but it is also possible to mix the mother liquor with the organic phase without subjecting the mother liquor to any further process steps before mixing with the organic phase.

Independently of being mixed before or after the phase separation, the mixture of organic phase and mother liquor or, alternatively, the organic phase is the second stream comprising the carboxylic acid which is purified in (c).

To reduce the amount of impurities recycled into the reaction (a) by circulating the carboxylic acid, purifying (c) of the second stream comprising carboxylic acid comprises stripping and distillation.

As the organic phase obtained in the phase separation usually still contains remainders of water, it is advantageous to further work up the organic phase to remove the water before reusing the organic phase. Besides the carboxylic acid as main component of the organic phase, the organic phase may comprise further impurities like by-products of the chemical reaction and the purification step of the DCDPS. Additionally, also impurities of the input streams may be comprised in the organic phase. These impurities for example include solvent in the used DCDPSO, particularly monochlorobenzene. “Input streams” here denote all components which are fed into the process, particularly the DCDPSO, the carboxylic acid and the oxidizing agent used for the reaction. Further input streams are the aqueous base and the water used for washing the DCDPS.

The impurities in the organic phase differ in low boiling impurities and high boiling impurities. Low boiling impurities are those impurities which have a boiling point below the boiling point of the carboxylic acid and high boiling impurities are those having a boiling point above the boiling point of the carboxylic acid. Typical low boiling impurities in the second stream comprising the carboxylic acid comprise at least one of water, monochlorobenzene, cyclic, linear and branched derivatives of the used carboxylic acid and depending on their chemical structure also lactones and linear or branched C₅ to C₇ alkanes. From these low boiling impurities, typically water and monochlorobenzene can be removed by stripping. The further low boiling impurities as well as remainders of water and monochlorobenzene which were not removed by stripping can be removed at least partly by the distillation.

Typical high boiling impurities are by-products of the chemical reaction and further also impurities which might be introduced into the process with the components fed into the process. Typical high boiling impurities comprise at least one of lactone, linear or branched C₅ to C₇ alkanes, isomers of DCDPS and isomers of 4,4′-dichlorodiphenyl sulfoxide, sulfuric acid, aluminum chloride, sodium sulphate and sodium hydroxide.

To remove the low boiling impurities comprising water and monochlorobenzene, in the first and third alternatives, the second stream comprising carboxylic acid is stripped in total in (a1 and c3, respectively) with a stripping gas. In the second alternative, the second stream comprising carboxylic acid is separated into a first part and a second part and only the first part is subjected to stripping in (b2).

In contrast to a distillation, by stripping also small amounts of the low boilers can be removed. This allows to achieve a purified carboxylic acid comprising less than 1.5 wt %, preferably less than 1 wt % and particularly less than 0.6 wt % of water and less than 1.5 wt %, preferably less than 1 wt % and particularly less than 0.6 wt % of monochlorobenzene, each based on the total amount of the purified carboxylic acid recycled into the reaction (a).

Independently of whether the second stream comprising carboxylic acid is subjected to stripping in total or only a part of the second stream comprising carboxylic acid is subjected to stripping, stripping preferably is carried out at a temperature in the range from 80 to 100° C., more preferred at a temperature in the range from 85 to 95° C. and particularly at a temperature in the range from 85 to 90° C. and a pressure in the range from 0.1 to 0.7 bar(abs), more preferred in the range from 0.2 to 0.4 bar(abs) and particularly in the range from 0.25 to 0.35 bar(abs).

In the stripping process, a stripping gas flows through the second stream. The stripping gas is selected such that it is inert towards the components comprised in the second stream. A suitable stripping gas preferably is nitrogen, a noble gas, carbon dioxide, or a mixture thereof. Particularly preferably, the stripping gas is nitrogen.

Stripping can be carried out in any apparatus suitable for a stripping process and known to a skilled person. Usually stripping is carried out in a stripping column in which the liquid phase—according to the present invention at least a part of the second stream comprising carboxylic acid—and the stripping gas flow in counter current. Preferably, a column is used into which the liquid phase is added at the top and the stripping gas at the bottom. To achieve an intimate mixing of the liquid phase and the stripping gas, the stripping column may comprise internals, for example a structured packing, a random packing or trays. Particularly, the internals in the stripping column are a random packing or structured packing.

To further purify the carboxylic acid and to avoid accumulation of impurities, according to the invention in the first alternative after stripping the crude carboxylic acid obtained in the stripping (a1) is separated into a first carboxylic acid stream and a second carboxylic acid stream. The second carboxylic acid stream then is fed into a distillation (c1).

In the second and third alternatives, to remove high boilers, the second part directly is fed into the distillation without previous stripping of low boilers.

It has been shown that for removing high boiling impurities and low boiling impurities in such an amount that no accumulation occurs, only a part of the crude carboxylic acid must be subjected to the distillation. This has the advantage, that energy can be saved and a smaller apparatus for the distillation can be used.

If the total second stream comprising the carboxylic acid is stripped and the thus obtained crude carboxylic acid is separated into a first and a second carboxylic acid stream, the second carboxylic acid stream which is fed into the distillation preferably contains 2 to 25 vol % of the crude carboxylic acid. More preferred, the second carboxylic acid stream contains 5 to 20 vol % and particularly 7 to 15 vol % of the crude carboxylic acid and the first carboxylic acid stream the rest of the crude carboxylic acid.

If the second stream comprising carboxylic acid is separated into a first part and a second part which is subjected to distillation according to the second and third alternatives, the second part which is fed into the distillation comprises 2 to 25 vol %, more preferred 5 to 20 vol % and particularly 7 to 15 vol % of the second stream comprising carboxylic acid and the first part the rest of the second stream comprising carboxylic acid.

The distillation of the second carboxylic acid stream or the second part of the second stream comprising the carboxylic acid can be carried out in any apparatus suitable for carrying out a distillation and which allows to withdraw a stream comprising high boilers, a stream comprising low boilers and a stream comprising at least one component having a boiling temperature between the boiling point of high boilers and low boilers. Usually, such an apparatus for carrying out the distillation is a distillation column. From the distillation column, the low boilers are withdrawn as top stream, the high boilers as bottom stream and the at least one component having a boiling temperature between the boiling point of high boilers and low boilers as a side stream. For purification of the second carboxylic acid stream, the side stream comprises the purified carboxylic acid, the bottom stream comprises high boiling impurities and the top stream low boiling impurities.

If a distillation column is used for carrying out the distillation, the distillation column preferably comprises internals. The internals used in the distillation columns can be any internals usually used in distillation columns, for example structured packings, random packings, trays or at least two of these internals, for example one or two random packings and at least one tray. If trays are used in the distillation column, any trays known to a skilled person can be used, for example perforated plates, bubble cap trays, sieve trays, or valve trays. However particularly preferred as internals are random packings, for example Raschig® Superrings 0.6 which show an optimal volume to pressure loss performance for the distillation to remove the high boilers.

The distillation preferably is carried out at a bottom temperature in a range from 130 to 250° C., more preferred in a range from 150 to 220° C. and particularly in a range from 190 to 215° C., a top temperature in the range from 50 to 150° C., more preferred from 100 to 140° C. and particularly in a range from 120 to 140° C., and a pressure in a range from 10 mbar(abs) to 400 mbar(abs), more preferred in a range from 20 mbar(abs) to 300 mbar(abs) and particularly in a range from 30 mbar(abs) to 250 mbar(abs).

The carboxylic acid withdrawn from the distillation column as side stream is mixed with the first carboxylic acid stream and recycled into the reaction (a). Depending on whether the reaction is carried out continuously or batchwise, it is possible, that the purified carboxylic acid cannot be recycled directly into the reaction (a). Therefore, it is preferred to collect the purified carboxylic acid in a buffer vessel before recycling it into the reaction (a). By collecting the purified carboxylic acid in the buffer vessel, it is possible to take the carboxylic acid from the buffer vessel in an amount as needed for the reaction and at a time, when it is needed. Further, using a buffer vessel allows to balance variations if such variations occur.

To keep the reaction temperature essentially constant and further to avoid heating of the components used for the reaction in the reactor, it is preferred to temper the purified carboxylic acid to a temperature in the range from 80 to 100° C. and particularly in a range between 80 and 100° C. before recycling it into the reaction. This temperature corresponds to the temperature at which the reaction is carried out and thus it is not necessary to heat a huge amount of components in the reactor before the reaction starts.

An illustrative embodiment of the invention is shown in the figure and explained in more detail in the following description.

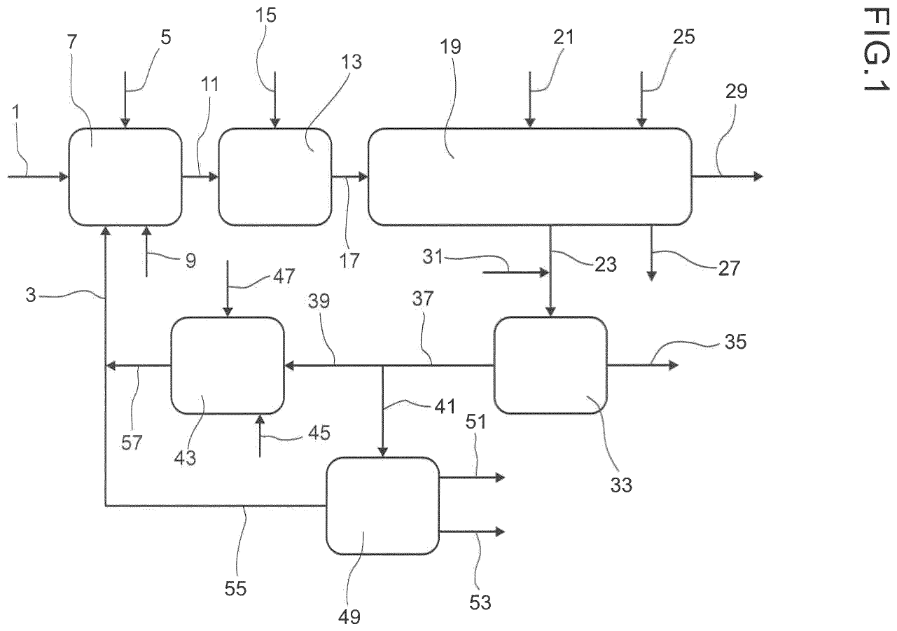

FIG. 1 shows a flow diagram of an embodiment of the inventive process.

In a process for producing DCDPS, 4,4′-dichlorodiphenyl sulfoxide 1, carboxylic acid 3, particularly heptanoic acid, and an oxidant 5, for example hydrogen peroxide are fed into an oxidation reactor 7. The oxidation reaction preferably is carried out in an inert atmosphere. To obtain the inert atmosphere, an inert gas 9, for example nitrogen, is fed into the oxidation reactor 7. In the oxidation reactor, the 4,4′-dichlorodiphenyl sulfoxide is oxidized forming DCDPS. This reaction preferably is carried out in the presence of an acidic catalyst. The acidic catalyst for example is a strong inorganic acid like sulfuric acid or a strong organic acid like methane sulfonic acid, or a mixture of at least two strong acids. The acidic catalyst also is fed into the oxidation reactor 7. To feed the acidic catalyst into the oxidation reactor 7, it is for example possible to feed the acidic catalyst via a separate feed line or to mix the catalyst with any of the other components added to the oxidation reactor 7, for example the 4,4′-dichlorodiphenyl sulfoxide 1, the oxidant 5 or the carboxylic acid 3. If the acidic catalyst is mixed with one of the compounds fed into the oxidation reactor 7, it is most preferred to mix the acidic catalyst with the carboxylic acid 3 and add this mixture into the oxidation reactor 7 just before heating up to reaction temperature.

In the oxidation reactor 7 a reaction mixture 11 is formed comprising the DCDPS. This reaction mixture 11 is withdrawn from the oxidation reactor 7 and fed into a crystallization apparatus 13. In the crystallization apparatus 13 the reaction mixture 11 is cooled and the DCDPS starts to solidify and form crystals. By this process a suspension is formed comprising the solid DCDPS in a mother liquor, the mother liquor comprising carboxylic acid, non-crystallized DCDPS and further liquid by-products and non-reacted reactants of the oxidization reaction.

Preferably, for cooling the reaction mixture to crystallize the DCDPS, water 15 is added to the crystallization apparatus 13. Then the pressure is reduced that water starts to evaporate. The evaporated water is cooled to condense, and the condensed water is returned into the crystallization apparatus 13. By this process, the reaction mixture is cooled without using cooled surfaces on which crystallized DCDPS can deposit and form solid deposits which have to be removed in a cleaning process.

The suspension 17 formed in the crystallization apparatus 13 then is fed into a solid-liquid separation apparatus 19. In the solid-liquid separation apparatus 19 first mother liquor is filtrated off the solid DCDPS whereby moist DCDPS is obtained. After the solid-liquid separation, in a first washing phase, an aqueous base 21 is added to the moist DCDPS. In this first washing phase the anion of the carboxylic acid reacts with the cation of the aqueous base forming an organic salt. The main portion of the organic salt formed by this reaction is removed from the filtration apparatus 19 with the aqueous base 23 which was used for washing. After washing with the aqueous base, in a second washing phase, the DCDPS is washed with water 25. Washing with water may be carried out in one or more steps. Washing with water thereby preferably is continued until the washed DCDPS is neutral which means a pH value in the range from 6.5 to 7.5. After being used for washing, the used water 27 is withdrawn from the process. It is particularly preferred as shown in the figure to carry out the solid-liquid separation and the washing in the same apparatus.

Washed DCDPS 29 is withdrawn from the solid-liquid separation apparatus 19 as product. The solid-liquid separation apparatus 19 can be any suitable filtration apparatus like an agitated pressure nutsche, a rotary pressure filter, a drum filter, a belt filter. Besides a filtration apparatus, the solid-liquid separation apparatus 19 also can be a centrifuge. Further, it is also possible to carry out the solid-liquid separation in one apparatus and the washing in a washing apparatus.

In the inventive process, after being withdrawn from the washing, the used aqueous base 23 is mixed with a strong acid 31. This mixture is fed into a phase separation 33 where an aqueous phase comprising a water-soluble salt and an organic phase comprising carboxylic acid is obtained. The aqueous phase 35 comprising salts which was obtained by the reaction of the aqueous base with the carboxylic acid and subsequently by the reaction with the strong acid 31 is withdrawn from the process.

The organic phase 37 which comprises the carboxylic acid in the following also is termed as “stream comprising carboxylic acid”. The stream 37 comprising the carboxylic acid is separated into a first part 39 and a second part 41.

The first part 39 is fed into a stripping apparatus 43, where low boilers are stripped from the first part of the stream comprising carboxylic acid. For stripping the low boilers, a stripping gas 45 is fed into the stripping apparatus 43. The stripping gas 45 preferably is nitrogen. By bringing the first part of the stream comprising carboxylic acid into contact with the inert gas, the low boilers, particularly water and solvent, for example monochlorobenzene, are at least partly separated from the carboxylic acid and mix with the nitrogen. The nitrogen with the low boilers then is withdrawn from the stripping apparatus 43 as flue gas 47. The carboxylic acid from which the low boilers are stripped is withdrawn from the stripping apparatus 43 as crude carboxylic acid 57.

The second part 41 of the stream comprising carboxylic acid is fed into a distillation 49. In the distillation, low boilers as well as high boilers are separated from the carboxylic acid. The low boilers are removed from the distillation 49 as top stream 51 and the high boilers as bottom stream 53. The carboxylic acid is removed from the distillation 49 as a side stream 55.

The side stream 55 comprising the carboxylic acid and the crude carboxylic acid 57 withdrawn from the stripping apparatus 43 are mixed and returned into the oxidation reactor 7 as carboxylic acid 3.

The process conditions for carrying out the process for producing DCDPSO correspond to those as described above.

### EXAMPLES

### Example 1

Distillation

310 g of the mother liquor as second stream comprising carboxylic acid obtained in an oxidation/crystallization process for producing DCDPS were fed into a batch distillation column with 10 trays. The mother liquor had following composition:

0.715 wt % monochlorobenzene, 0.02 wt % dodecane, 0.003 wt % heptanoic acid methyl ester, 0.026 wt % valeric acid, 0.315 wt % hexanoic acid, 95.02 wt % heptanoic acid and 3.5 wt % water. This mother liquor was distilled with a bottom temperature of 160° C., and a top temperature of 135° C. at a pressure of 52 mbar (abs) for about 4.5 h. The carboxylic acid obtained by this distillation had the following composition:

0.014 wt % monochlorobenzene, 0.002 wt % dodecane, 0.0 wt % heptanoic acid methyl ester, 0.005 wt % valeric acid, 0.185 wt % hexanoic acid, and 99.52 wt % heptanoic acid.

Stripping

2627 g of the mother liquor as second stream comprising carboxylic acid obtained in an oxidation/crystallization process with a temperature of 88° C. were provided in a buffer vessel and continuously fed into a stripping column with a feed rate of 66 ml/min.

The mother liquor had the following composition:

0.715 wt % monochlorobenzene, 0.02 wt % dodecane, 0.003 wt % heptanoic acid methyl ester, 0.026 wt % valeric acid, 0.315 wt % hexanoic acid, 95.02 wt % heptanoic acid and 3.5 wt % water.

The stripping column had 10 trays and the mother liquor was fed on top into the stripping column and 150 NL per hour nitrogen were fed into the stripping column at the bottom as stripping gas. The pressure in the stripping column was set to 300 mbar.

After stripping, the carboxylic acid was continuously removed from the stripping column and had the following composition:

0.456 wt % monochlorobenzene, 0.018 wt % dodecane, 0.003 wt % heptanoic acid methyl ester, 0.025 wt % valeric acid, 0.333 wt % hexanoic acid, 95.36 wt % heptanoic acid, and 0.42 wt % water.

The carboxylic acid obtained by distillation and the carboxylic acid obtained by stripping were mixed and the resulting purified carboxylic acid contained 0.41 wt % monochlorobenzene, 2.2 wt % 4,4′-DCDPS, 0.54% 2,4′-DCDPS, about 600 ppm lactones, 4000 ppm n-hexanoic acid, 240 ppm valerian acid, 100 ppm esters, and 160 ppm dodecane.

### Example 2

Distillation

311 g of the mother liquor as second stream comprising carboxylic acid obtained in an oxidation/crystallization process for producing DCDPS were fed into a batch distillation column with 10 trays. The mother liquor contained:

0.696 wt % monochlorobenzene, 0.016 wt % dodecane, 0.024 wt % valeric acid, 0.292 wt % hexanoic acid, 2.6 wt % DCDPS, 95.006 wt % heptanoic acid. The remainders were further impurities, particularly water.

This mother liquor was distilled with a bottom temperature of 160° C., and a top temperature of 135° C. at a pressure of 52 mbar (abs) for about 4.5 h. The energy consumption for distillation was about 465 kJ steam per kg DCDPS produced.

The carboxylic acid obtained by this distillation had the following composition:

210 ppm monochlorobenzene, 10 ppm dodecane, 30 ppm valeric acid, 1500 ppm hexanoic acid, and 99.67 wt % heptanoic acid.

The bottom stream of the distillation contained about 71 wt % heptanoic acid and about 20 wt % DCDPS. Due to the temperature in the bottom stream the DCDPS changed its color and therefore the bottom stream was disposed.

Stripping

2608 g of the mother liquor with the same composition as described above for the mother liquor fed into the distillation were provided in a buffer vessel with a temperature which was kept in a range between 78° C. and 86° C. and continuously fed into a stripping column with a feed rate of 66 ml/min.

The stripping column had 10 trays and the mother liquor was fed on top into the stripping column and 150 NL per hour nitrogen were fed into the stripping column at the bottom as stripping gas. The pressure in the bottom of the stripping column was set to 300 mbar.

After stripping, the carboxylic acid was continuously removed from the stripping column and had the following composition:

0.484 wt % monochlorobenzene, 0.015 wt % dodecane, 0.022 wt % valeric acid, 0.305 wt % hexanoic acid, 2.4 wt % DCPDS and 95.47 wt % heptanoic acid.

The combined carboxylic acid was recycled in the production of DCDPS.

As only the bottom stream of the distillation was disposed and the combined carboxylic acid obtained in the distillation and stripping processes was recycled into the production of DCDPS, the amount of DCDPS which was lost was 0.38 wt % of the DCDPS produced and the amount of heptanoic acid which was lost was 0.47 wt % of the heptanoic acid used in the process.

1.-18. (canceled)

19. A process for producing 4,4′-dichlorodiphenyl sulfone, comprising:
(a) reacting 4,4′-dichlorodiphenyl sulfoxide and an oxidizing agent in at least one carboxylic acid as solvent to obtain a reaction mixture comprising 4,4′-dichlorodiphenyl sulfone and the at least one carboxylic acid;
(b) separating the reaction mixture into a first stream comprising 4,4′-dichlorodiphenyl sulfone and a second stream comprising the at least one carboxylic acid;
(c) purifying the second stream comprising the at least one carboxylic acid by
distilling a part of the second stream comprising the at least one carboxylic acid;
stripping low boilers from at least a part of the second stream comprising the at least one carboxylic acid;
(d) recycling the purified at least one carboxylic acid into the reaction (a).

20. The process according to claim 19, wherein purifying the second stream comprising the at least one carboxylic acid comprises:
(a1) stripping low boilers from the second stream comprising the at least one carboxylic acid in a stripping column using a stripping gas to obtain a crude carboxylic acid;
(b1) separating the crude carboxylic acid into a first carboxylic acid stream and a second carboxylic acid stream;
(c1) distilling the second carboxylic acid stream to obtain a bottom stream comprising high boiling impurities, a top stream comprising low boiling impurities and a side stream comprising the at least one carboxylic acid; and
(d1) mixing the first carboxylic acid stream and the side stream to obtain the purified at least one carboxylic acid.

21. The process according to claim 19, wherein purifying the second stream comprising the at least one carboxylic acid comprises:
(a2) separating the second stream comprising the at least one carboxylic acid into a first part and a second part;
(b2) stripping low boilers from the first part comprising the at least one carboxylic acid in a stripping column using a stripping gas to obtain a crude carboxylic acid;
(c2) distilling the second part to obtain a bottom stream comprising high boiling impurities, a top stream comprising low boiling impurities and a side stream comprising the at least one carboxylic acid; and
(d2) mixing the crude carboxylic acid obtained in (b2) and the side stream comprising the at least one carboxylic acid obtained in (c2) to obtain the purified at least one carboxylic acid.

22. The process according to claim 19, wherein purifying the second stream comprising the at least one carboxylic acid comprises:
(a3) separating the second stream comprising the at least one carboxylic acid into a first part and a second part;
(b3) distilling the second part to obtain a bottom stream comprising high boiling impurities, a top stream comprising low boiling impurities and a side stream comprising the at least one carboxylic acid; and
(c3) mixing the side stream comprising the at least one carboxylic acid obtained in (b3) and the first part of the second stream comprising the at least one carboxylic acid to obtain crude carboxylic acid;
(d3) stripping low boilers from the crude carboxylic acid obtained in (c3) in a stripping column using a stripping gas to obtain the purified at least one carboxylic acid.

23. The process according to claim 19, wherein the oxidizing agent is a peroxide.

24. The process according to claim 19, wherein the low boilers stripped from the second stream comprising the at least one carboxylic acid and the low boiling impurities comprise at least one of water and monochlorobenzene.

25. The process according to claim 19, wherein the part of the second stream comprising the carboxylic acid which is distilled contains 2 to 25 vol % of the second stream comprising the carboxylic acid.

26. The process according to claim 19, wherein the high boiling impurities comprise at least one of lactone, linear or branched C₄ to C₇ alkanes.

27. The process according to claim 19, wherein stripping the low boilers from the second stream comprising the at least one carboxylic acid is performed at a temperature in the range from 80 to 100° C. and a pressure in the range from 0.1 to 0.7 bar(abs).

28. The process according to claim 19, wherein the stripping gas is nitrogen, a noble gas, carbon dioxide, or a mixture thereof.

29. The process according to claim 19, wherein distilling of the part of the second stream comprising the at least one carboxylic acid is carried out at a bottom temperature in the range from 130 to 250° C., a top temperature in the range from 50 to 150° C. and a pressure in the range from 10 to 400 mbar (abs).

30. The process according to claim 19, wherein the purified at least one carboxylic acid is collected in a buffer vessel before being recycled into the reaction (a).

31. The process according to claim 19, wherein the purified at least one carboxylic acid is tempered to a temperature in the range between 80 and 100° C. before being recycled into the reaction (a).

32. The process according to claim 19, wherein for separating the reaction mixture into a first stream comprising 4,4′-dichlorodiphenyl sulfone and a second stream comprising the at least one carboxylic acid, the reaction mixture is cooled to a temperature where the 4,4′-dichlorodiphenyl sulfone crystallizes to obtain a suspension comprising the crystallized 4,4′-dichlorodiphenyl sulfone and a liquid phase and the suspension is separated by a solid-liquid separation into residual moisture comprising 4,4′-dichlorodiphenyl sulfone and mother liquor.

33. The process according to claim 32, wherein the residual moisture comprising 4,4′-dichlorodiphenyl sulfone is washed with an aqueous base and subsequently with water.

34. The process according to claim 33, wherein the aqueous base after being used for washing is mixed with a strong acid by which an aqueous phase comprising a water soluble salt and an organic phase comprising the at least one carboxylic acid is obtained.

35. The process according to claim 34, wherein the organic phase comprising the at least one carboxylic acid is mixed with the mother liquor to obtain the second stream comprising the at least one carboxylic acid or wherein the mother liquor and the aqueous base are mixed before carrying out a phase separation to obtain the aqueous phase comprising a salt and the organic phase comprising the at least one carboxylic acid, the organic phase comprising the at least one carboxylic acid being the second stream comprising the at least one carboxylic acid.

36. The process according to claim 19, wherein the at least one carboxylic acid is n-hexanoic acid, n-heptanoic acid or a mixture thereof.
